SEQUENCE LISTING (1 sequence)

SEQ ID NO: 1 - DNA, 26 bp, Artificial Sequence
  Synthetic DNA polynucleotide
ggtggtggtggtgttggtggtggtgg

(19) United States
(12) Patent Application Publication
(10) Pub. No.: US 2012/0082616 A1
(43) Pub. Date: Apr. 5, 2012

(54) Aptamer Conjugates for Targeting of Therapeutic and/or Diagnostic Nanocarriers

(75) Inventors: Bobby N. Trawick, Florissant, MO (US); Todd A. Osiek, Ballwin, MO (US); James R. Wheatley, JR., St. Louis, MO (US)

(73) Assignee: Mallinckrodt LLC, Hazelwood, MO (US)

(21) Appl. No.: 13/241,368

(22) Filed: Sep. 23, 2011

Related U.S. Application Data
(60) Provisional application No. 61/386,201, filed on Sep. 24, 2010.

(51) Int. Cl. A61K 9/127; A61K 33/24; A61K 31/282; A61K 31/513; A61K 31/7068; A61K 51/02; A61K 49/08; A61K 49/18; A61K 49/04; A61K 31/704; A61K 51/12; B82Y 40/00; B82Y 99/00; B82Y 5/00

(52) U.S. Cl. 424/1.21; 514/34; 424/649; 514/492; 514/274; 514/49; 424/1.61; 424/9.6; 424/9.3; 424/9.4; 424/450; 977/774; 977/797; 977/801; 977/907; 977/928; 977/930; 977/915; 977/927; 977/840

(57) ABSTRACT

The present invention provides targeted delivery compositions and their methods of use in treating and diagnosing a disease state in a subject.

### CROSS-REFERENCES TO RELATED APPLICATIONS

This application claims the benefit of U.S. Provisional Application No. 61/386,201 filed Sep. 24, 2010, which is incorporated herein in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cancer is a class of diseases that can affect people of all ages. Accordingly, there is considerable effort to provide therapies that can treat or diagnose cancer in patients. Targeted delivery of nanocarriers in the body has been discussed recently as a potential new avenue in drug delivery and diagnostic imaging techniques. Unfortunately, obstacles still exist in making nanocarrier based-products that can effectively treat or diagnose cancer. Thus, there is a need for new targeted delivery approaches that can treat or diagnose cancer and provide ways to facilitate personalized care for a patient.

### BRIEF SUMMARY OF THE INVENTION

The present invention provides targeted delivery compositions and their methods of use in treating and diagnosing a disease state, such as a cancerous condition, in a subject.

In an aspect of the invention, the targeted delivery compositions can include a nanocarrier including a therapeutic agent, a diagnostic agent, or a combination thereof, and a conjugate having the formula: A-[(EG)(P)]ₙ-T, each of which is described in more detail below. In another aspect, the targeted delivery compositions can include a conjugate having the formula: (DT)-[(EG)(P)]ₘ-T, which is described in more detail below.

The targeted delivery compositions and methods of making and using such compositions provide a number of unique aspects to the areas of drug delivery and diagnostic imaging. For example, the targeted delivery compositions linking groups that can be synthesized to have a discrete number of monomers, which can be tailored to, e.g., provide a specific length and/or chemical property. Furthermore, the monomers making up the linking groups are fully customizable and can be prepared to include only one type of monomer or multiple types of monomers in any order. The linking groups can also be synthesized on a solid phase support, which allows for simple, automated syntheses. In addition to the linking groups, the targeted delivery compositions can be used to treat diseases more effectively by utilizing lower doses of agents that if administered with normal dosage amounts might otherwise be toxic to a patient.

A further understanding of the nature and advantages of the present invention can be realized by reference to the remaining portions of the specification and the drawings.

### BRIEF DESCRIPTION OF THE DRAWINGS

FIG. 1 depicts a generalized aptamer-(HFGp)ₙ-cholesterol conjugate in accordance with an exemplary embodiment of the invention.

FIG. 2 shows an example of an aptamer-(HEGp)ₙ-cholesterol targeted liposome in accordance with an exemplary embodiment of the invention.

FIG. 3 illustrates an AS1411-(HEGp)₈-cholesterol conjugate, in accordance with an exemplary embodiment of the invention.

FIG. 4 illustrates (A) a HPLC trace of semi-preparative injection of crude AS1411-(HEGp)₈-cholesterol conjugate, (B) a HPLC of crude AS1411-(HEGp)₈-cholesterol conjugate, and (C) a HPLC of purified AS1411-(HEGp)₈-cholesterol conjugate, in accordance with exemplary embodiments of the invention.

FIG. 5 shows a Total Ion Current and Mass Spectrum of Purified AS1411-(HEGp)₈-cholesterol, in accordance with exemplary embodiments of the invention.

### DETAILED DESCRIPTION OF THE INVENTION

### I. Definitions

As used herein, the term “targeted delivery composition” refers to both a composition of a nanocarrier attached to a conjugate having the formula: A-[(EG)(P)]ₙ-T, or a conjugate having the formula: (DT)-[(EG)(P)]ₘ-T that is not attached to a nanocarrier, as further described herein. The compositions of the present invention can be used as therapeutic compositions, as diagnostic compositions, or as both therapeutic and diagnostic compositions. In certain embodiments, the compositions can be targeted to a specific target within a subject or a test sample, as described further herein.

As used herein, the term “nanocarrier” refers to particles of varied size, shape, type and use, which are further described herein. As will be appreciated by one of ordinary skill in the art, the characteristics of the nanocarriers, e.g., size, can depend on the type and/or use of the nanocarrier as well as other factors generally well known in the art. In general, nanocarriers can range in size from about 1 nm to about 1000 nm. In other embodiments, nanocarriers can range in size from about 10 nm to about 200 nm. In yet other embodiments, nanocarriers can range in size from about 50 nm to about 150 nm. In certain embodiments, the nanocarriers are greater in size than the renal excretion limit, e.g., greater than about 6 nm in diameter. In other embodiments, the nanocarriers are small enough to avoid clearance from the bloodstream by the liver, e.g., smaller than 1000 nm in diameter. Nanocarriers can include spheres, cones, spheroids, and other shapes generally known in the art. Nanocarriers can be hollow (e.g., solid outer core with a hollow inner core) or solid or be multilayered with hollow and solid layers or a variety of solid layers. For example, a nanocarrier can include a solid core region and a solid outer encapsulating region, both of which can be cross-linked. Nanocarriers can be composed of one substance or any combination of a variety of substances, including lipids, polymers, magnetic materials, or metallic materials, such as silica, gold, iron oxide, and the like. Lipids can include fats, waxes, sterols, cholesterol, fat-soluble vitamins, monoglycerides, diglycerides, phospholipids, sphingolipids, glycolipids, cationic or anionic lipids, derivatized lipids, cardiolipin and the like. Polymers can include block copolymers generally, poly(lactic acid), poly(lactic-co-glycolic acid), polyethylene glycol, acrylic polymers, cationic polymers, as well as other polymers known in the art for use in making nanocarriers. In some embodiments, the polymers can be biodegradable and/or biocompatible. Nanocarriers can include a liposome, a micelle, a lipoprotein, a lipid-coated bubble, a block copolymer micelle, a polymersome, a noisome, a quantum dot, an iron oxide particle, a gold particle, a dendrimer, or a silica particle. In certain embodiments, a lipid monolayer or bilayer can fully or partially coat a nanocarrier composed of a material capable of being coated by lipids, e.g., polymer nanocarriers. In some embodiments, liposomes can include multilamellar vesicles (MLV), large unilamellar vesicles (LUV), and small unilamellar vesicles (SUV).

As used herein, the term “therapeutic agent” refers to a compound or molecule that, when present in an effective amount, produces a desired therapeutic effect on a subject in need thereof. The present invention contemplates a broad range of therapeutic agents and their use in conjunction with the targeted delivery compositions, as further described herein.

As used herein, the term “diagnostic agent” refers to a component that can be detected in a subject or test sample and is further described herein.

As used herein, the term “conjugate” refers generally to a molecule that includes a linking group. In some embodiments, a conjugate of the present invention has the formula: A-[(EG)(P)]ₙ-T. A is an attachment component that can attach (covalently or non-covalently) the conjugate to a nanocarrier. The conjugate can be covalently bonded to any part of a nanocarrier including the surface or an internal region. Covalent attachment can be achieved through a functional group using a linking chemistry well known in the art, which is further described herein. In other embodiments, a non-covalent attachment can include interactions that are generally well known in the art and further described herein. The conjugates of the present invention can further include a linking group having the formula [(EG)(P)]ₙ and a targeting agent, T, each being described further herein. In other embodiments, a conjugate of the present invention can include a targeted delivery composition having the formula (DT)-[(EG)(P)]ₘ-T, which is described further below.

As used herein, the term “linking group” refers to part of a conjugate that links two components, e.g., an attachment component and a targeting agent. Depending on the conjugate being prepared and the properties desired for the conjugate, the linking group can be assembled from readily available Monomeric components to achieve an appropriate separation of targeting agent and nanocarrier or agent.

As used herein, the term “targeting agent” refers to a molecule that is specific for a target. In certain embodiments, a targeting agent can include a small molecule mimic of a target ligand (e.g., a peptide mimetic ligand), a target ligand (e.g., an RGD peptide containing peptide or folate amide), or an antibody or antibody fragment specific for a particular target. Targeting agents can bind a wide variety of targets, including targets in organs, tissues, cells, extracellular matrix components, and/or intracellular compartments that can be associated with a specific developmental stage of a disease. In some embodiments, targets can include cancer cells, particularly cancer stem cells. Targets can further include antigens on a surface of a cell, or a tumor marker that is an antigen present or more prevalent on a cancer cell as compared to normal tissue. In certain embodiments, a targeting agent can further include folic acid derivatives, B-12 derivatives, integrin RGD peptides, RGD mimetics, NGR derivatives, somatostatin derivatives or peptides that bind to the somatostatin receptor, e.g., octreotide and octreotate, and the like. In some embodiments, a targeting agent can be an aptamer—which is composed of nucleic acids (e.g., DNA or RNA), or a peptide and which binds to a specific target. A targeting agent can be designed to bind specifically or non-specifically to receptor targets, particularly receptor targets that are expressed in association with tumors. Examples of receptor targets include, but are not limited to, MUC-1, EGFR, Claudin 4, MUC-4, CXCR4, CCR7, FOL1R, somatostatin receptor 4, Erb-B2 (erythroblastic leukaemia oncogene homologue 2) receptor, CD44 receptor, and VEGF receptor-2 kinase.

As used herein, the term “stealth agent” refers to a molecule that can modify the surface properties of a nanocarrier. A stealth agent can prevent nanocarriers from sticking to each other and to blood cells or vascular walls. In certain embodiments, stealth nanocarriers, e.g., stealth liposomes, can reduce immunogenicity and/or reactogenecity when the nanocarriers are administered to a subject. Stealth agents can also increase blood circulation time of a nanocarrier within a subject. In some embodiments, a nanocarrier can include a stealth agent such that, for example, the nanocarrier is partially or fully composed of a stealth agent or the nanocarrier is coated with a stealth agent. Stealth agents for use in the present invention can include those generally well known in the art. In certain embodiments, a stealth agent can include “polyethylene glycol,” which is well known in the art and refers generally to an oligomer or polymer of ethylene oxide. Polyethylene glycol (PEG) can be linear or branched, wherein branched PEG molecules can have additional PEG molecules emanating from a central core and/or multiple PEG molecules can be grafted to the polymer backbone. PEG can include low or high molecular weight PEG, e.g., PEG500, PEG2000, PEG3400, PEG5000, PEG10000, or PEG20000 wherein the number, e.g., 500, indicates the average molecular weight. In certain embodiments, PEGylated-lipids are present in a bilayer of the nanocarrier, e.g., a liposome, in an amount sufficient to make the nanocarrier “stealth,” wherein a stealth nanocarrier shows reduced immunogenicity. Other suitable stealth agents can include but are not limited to dendrimers, polyalkylene oxide, polyvinyl alcohol, polycarboxylate, polysaccharides, and/or hydroxyalkyl starch. Stealth agents can be attached to the targeted delivery compositions of the present invention through covalent and/or non-covalent attachment, as described further herein.

As used herein, the term “embedded in” refers to the location of an agent on or in the vicinity of the surface of a nanocarrier. Agents embedded in a nanocarrier can, for example, be located within a bilayer membrane of a liposome or located within an outer polymer shell of a nanocarrier so as to be contained within that shell.

As used herein, the term “encapsulated in” refers to the location of an agent that is enclosed or completely contained within the inside of a nanocarrier. For liposomes, for example, therapeutic and/or diagnostic agents can be encapsulated so as to be present in the aqueous interior of the liposome. Release of such encapsulated agents can then be triggered by certain conditions intended to destabilize the liposome or otherwise effect release of the encapsulated agents.

As used herein, the term “tethered to” refers to attachment of one component to another component so that one or more of the components has freedom to move about in space. In certain exemplary embodiments, an attachment component can be tethered to a nanocarrier so as to freely move about in solution surrounding the nanocarrier. In some embodiments, an attachment component can be tethered to the surface of a nanocarrier, extending away from the surface.

As used herein, the term “lipid” refers to lipid molecules that can include fats, waxes, sterols, cholesterol, fat-soluble vitamins, monoglycerides, diglycerides, phospholipids, sphingolipids, glycolipids, cationic or anionic lipids, derivatized lipids, and the like. Lipids can form micelles, monolayers, and bilayer membranes. In certain embodiments, the lipids can self-assemble into liposomes. In other embodiments, the lipids can coat a surface of a nanocarrier as a monolayer or a bilayer.

As used herein, the term “aptamer” refers to a non-naturally occurring oligonucleotide (typically 20-200 nucleotides) that specifically binds to a particular target. “Non-naturally occurring” encompasses non-naturally occurring sequences of natural nucleotides (A, T, C, G, U), as well as oligonucleotides with non-naturally occurring or modified nucleotides. For example, “Spiegelmers®” are aptamers with mirror image nucleic acids, i.e., in the L chiral configuration instead of the naturally occurring D configuration. Aptamers can form unique three-dimensional structures via intramolecular interactions, and/or change structure upon binding to a target, e.g., via an induced-fit mechanism from a primary or secondary structure. Aptamer binding to the target is not mediated by traditional complementary nucleic acid hybridization, e.g., double or triple helix formation, though portions of the aptamer may participate in such hybridization. For example, aptamers commonly form intramolecular hairpin structures and other three dimensional structures. Aptamers can be selected according to any method or combination of methods. Systematic Evolution of Ligands by Exponential Enrichment (SELEX™), or a variation thereof, is commonly used in the field. The basic SELEX™ process is described e.g., in U.S. Pat. No. 5,567,588. A number of variations on the basic method can also be used, e.g., in vivo SELEX™, as described in US Appl. No. 2010015041. MONOLEX™ is another selection process described, e.g., in Nitsche et al. (2007) BMC Biotechnology 7:48 and WO02/29093. In vivo selection using nucleic acid libraries injected into tumor cells is also possible (see, e.g., Mi et al., (2010) Nat. Chem. Biol. 1:22). Aptamers for use in the present invention can be designed to bind to a variety of targets, including but not limited to MUC-1, EGFR, Claudin 4, MUC-4, CXCR4, CCR7, FOL1R, somatostatin receptor 4, Erb-B2 (erythroblastic leukaemia oncogene homologue 2) receptor, CD44 receptor, VEGF receptor-2 kinase, and nucleolin.

As used herein, the term “subject” refers to any mammal, in particular human, at any stage of life.

As used herein, the terms “administer,” “administered,” or “administering” refers to methods of administering the targeted delivery compositions of the present invention. The targeted delivery compositions of the present invention can be administered in a variety of ways, including topically, parenterally, intravenously, intradermally, intramuscularly, colonically, rectally or intraperitoneally. Parenteral administration and intravenous administration are the preferred methods of administration. The targeted delivery compositions can also be administered as part of a composition or formulation.

As used herein, the terms “treating” or “treatment” of a condition, disease, disorder, or syndrome includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

As used herein, the term “formulation” refers to a mixture of components for administration to a subject. Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. The formulations of a targeted delivery composition can be presented in unit-dose or multi-dose sealed containers, such as ampoules and vials. A targeted delivery composition, alone or in combination with other suitable components, can be made into aerosol formulations (i.e., they can be “nebulized”) to be administered via inhalation through the mouth or the nose. Aerosol formulations can be placed into pressurized acceptable propellants, such as dichlorodifluoromethane, propane, nitrogen, and the like. Suitable formulations for rectal administration include, for example, suppositories, which comprises an effective amount of a targeted delivery composition with a suppository base. Suitable suppository bases include natural or synthetic triglycerides or paraffin hydrocarbons. In addition, it is also possible to use gelatin rectal capsules which contain a combination of the targeted delivery composition with a base, including, for example, liquid triglycerides, polyethylene glycols, and paraffin hydrocarbons. In certain embodiments, formulations can be administered topically or in the form of eye drops.

### Embodiments of the Invention

### II. General

The present invention provides targeted delivery compositions and their methods of use in treating and diagnosing a disease state in a subject. The disclosed compositions and methods provide a number of beneficial features over currently existing approaches. For example, the targeted delivery compositions include linking groups that can be synthesized to have a discrete number of monomers, which can be tailored to, e.g., provide a specific length and/or chemical property. Furthermore, the monomers making up the linking groups are fully customizable and can be prepared to include only one type of monomer or multiple types of monomers in any order. The linking groups can also be synthesized on a solid phase support, which allows for simple, automated syntheses. In addition to the linking groups, the targeted delivery compositions can be used to treat diseases more effectively by utilizing lower doses of agents that if administered with normal dosage amounts might otherwise be toxic to a patient.

### III. Targeted Delivery Compositions

### A. Targeted Delivery Compositions Including a Nanocarrier

In one aspect, the targeted delivery compositions of the present invention can include a targeted delivery composition, comprising: (a) a nanocarrier including a therapeutic or diagnostic agent or a combination thereof; and (b) a conjugate having the formula: A-[(EG)(P)]ₙ-T; wherein, A is an attachment component for attaching the conjugate to the nanocarrier; [(EG)(P)]ₙ is a linking group, wherein the subscript n is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate; and, T is a targeting agent

### Nanocarriers

A wide variety of nanocarriers can be used in constructing the targeted delivery compositions. As will be appreciated by one of ordinary skill in the art, the characteristics of the nanocarriers, e.g., size, can depend on the type and/or use of the nanocarrier as well as other factors generally well known in the art. Suitable particles can be spheres, spheroids, flat, plate-shaped, tubes, cubes, cuboids, ovals, ellipses, cylinders, cones, or pyramids. Suitable nanocarriers can range in size of greatest dimension (e.g., diameter) from about 1 nm to about 1000 nm, from about 10 nm to about 200 nm, and from about 50 nm to about 150 nm.

Suitable nanocarriers can be made of a variety of materials generally known in the art. In some embodiments, nanocarriers can include one substance or any combination of a variety of substances, including lipids, polymers, or metallic materials, such as silica, gold, iron oxide, and the like. Examples of nanocarriers can include but are not limited to a liposome, a micelle, a lipoprotein, a lipid-coated bubble, a block copolymer micelle, a polymersome, a noisome, an iron oxide particle, a gold particle, a silica particle, a dendrimer, or a quantum dot.

In some embodiments, the nanocarriers are liposomes composed partially or wholly of saturated or unsaturated lipids. Suitable lipids can include but are not limited to fats, waxes, sterols, cholesterol, fat-soluble vitamins, monoglycerides, diglycerides, phospholipids, sphingolipids, glycolipids, derivatized lipids, and the like. In some embodiments, suitable lipids can include amphipathic, neutral, non-cationic, anionic, cationic, or hydrophobic lipids. In certain embodiments, lipids can include those typically present in cellular membranes, such as phospholipids and/or sphingolipids. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Suitable sphingolipids include but are not limited to sphingosine, ceramide, sphingomyelin, cerebrosides, sulfatides, gangliosides, and phytosphingosine. Other suitable lipids can include lipid extracts, such as egg PC, heart extract, brain extract, liver extract, and soy PC. In some embodiments, soy PC can include Hydro Soy PC(HSPC). Cationic lipids include but are not limited to N,N-dioleoyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA). Non-cationic lipids include but are not limited to dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMFS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidylethanolamine (DPPE), dimyristoylphosphoethanolamine (DMPE), distearoyl-phosphatidyl-ethanolamine (DSPE), 16-O-monomethyl PE, 16-O-dimethyl PE, 1.8-1-trans PE, 1-stearoyl-2-oleoyl-phosphatidylethanolamine (SOPE),1,2-dielaidoyl-sn-glycero-3-phosphoethanolamine (transDOPE), and cardiolipin. In certain embodiments, the lipids can include derivatized lipids, such as PEGylated lipids. Derivatized lipids can include, for example, DSPE-PEG2000, cholesterol-PEG2000, DSPE-polyglycerol, or other derivatives generally well known in the art.

Any combination of lipids can be used to construct a nanocarrier, such as a liposome. In certain embodiments, the lipid composition of a targeted delivery composition, such as a Liposome, can be tailored to affect characteristics of the liposomes, such as leakage rates, stability, particle size, zeta potential, protein binding, in vivo circulation, and/or accumulation in tissue, such as a tumor, liver, spleen or the like. For example, DSPC and/or cholesterol can be used to decrease leakage from the liposomes. Negatively or positively lipids, such as DSPG and/or DOTAP, can be included to affect the surface charge of a Liposome. In some embodiments, the liposomes can include about ten or fewer types of lipids, or about five or fewer types of lipids, or about three or fewer types of lipids. In some embodiments, the molar percentage (mol %) of a specific type of lipid present typically comprises from about 0% to about 10%, from about 10% to about 30%, from about 30% to about 50%, from about 50% to about 70%, from about 70% to about 90%, from about 90% to 100% of the total lipid present in a nanocarrier, such as a liposome. The lipids described herein can be included in a liposome, or the lipids can be used to coat a nanocarrier of the invention, such as a polymer nanocarrier. Coatings can be partially or wholly surrounding a nanocarrier and can include monolayers and/or bilayers. In one embodiment, liposomes can be composed of about 50.6 mol HSPC, about 44.3 mol % cholesterol, and about 5.1 mol % DSPE-PEG2000.

In other embodiments, a portion or all of a nanocarrier can include a polymer, such as a block copolymer or other polymers known in the art for making nanocarriers. In some embodiments, the polymers can be biodegradable and/or biocompatible. Suitable polymers can include but are not limited to polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, and combinations thereof. In some embodiments, exemplary particles can include shell cross-linked kneels, which are further described in the following references: Becker et al., U.S. application Ser. No. 11/250,830; Thurmond, K. B. et al., J. Am. Chem. Soc., 119 (28) 6656-6665 (1997)); Wooley, K. L., Chem. Eur. J., 3 (9): 1397-1399 (1997); Wooley, K. L., J. Poly. Sci.: Part A: Polymer Chem., 38: 1397-1407 (2000). In other embodiments, suitable particles can include poly(lactic co-glycolic acid) (PLGA) (Fu, K. et al., Pharm Res., 27:100-106 (2000).

In yet other embodiments, the nanocarriers can be partially or wholly composed of materials that are metallic in nature, such as silica, gold, iron oxide, and the like. In some embodiments, the silica particles can be hollow, porous, and/or mesoporous (Slowing, I. I., et al., Adv. Drug Deliv. Rev., 60 (11):1278-1288 (2008)). Gold particles are generally known in the art, as provided by the following exemplary reference: Bhattacharya, R. & Mukherjee, P., Adv. Drug Deliv. Rev., 60(11): 1289-1306 (2008)). Iron oxide particles or quantum dots can also be used and are well-known in the art (van Vlerken, L. E. & Amiji, M. M., Expert Opin. Drug Deliv., 3(2): 205-216 (2006)). The nanocarriers also include but are not limited to viral particles and ceramic particles.

### Conjugates for Attaching to a Nanocarrier

In certain embodiments, the targeted delivery compositions including a nanocarrier also can include a conjugate having the formula: A-[(EG)(P)]ₙ-T, wherein the attachment component A can be used to attach the conjugate to a nanocarrier. The attachment component can attach to any location on the nanocarrier, such as on the surface of the nanocarrier. The attachment component can attach to the nanocarrier through a variety of ways, including covalent and/or non-covalent attachment. As described further below, the conjugate also includes a [(EG)(P)]ₙ linking group and a targeting agent, T.

In certain embodiments, the attachment component A can include a functional group that can be used to covalently attach the attachment component to a reactive group present on the nanocarrier. The functional group can be located anywhere on the attachment component, such as the terminal position of the attachment component. A wide variety of functional groups are generally known in the art and can be reacted under several classes of reactions, such as but not limited to nucleophilic substitutions (e.g., reactions of amines and alcohols with acyl halides or active esters), electrophilic substitutions (e.g., enamine reactions) and additions to carbon-carbon and carbon-heteroatom multiple bonds (e.g., Michael reaction or Diels-Alder addition). These and other useful reactions are discussed in, for example, March, Advanced Organic Chemistry, 3rd Ed., John Wiley & Sons, New York, 1985; and Hermanson, Bioconjugate Techniques, Academic Press, San Diego, 1996. Suitable functional groups can include, for example: (a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such reactions as Grignard addition or alkyl lithium addition; (f) sulfonyl halide groups for subsequent reaction with amines, for example, to form sulfonamides; (g) thiol groups, which can be converted to disulfides or reacted with acyl halides; (h) amine or sulfhydryl groups, which can be, for example, acylated, alkylated or oxidized; (i) alkenes, which can undergo, for example, cycloadditions, acylation, Michael addition, etc; and (j) epoxides, which can react with, for example, amines and hydroxyl compounds. In some embodiments, click chemistry-based platforms can be used to attach the attachment component to a nanocarrier (Kolb, H. C. et al. M. G. Finn and K. B. Sharpless, Angew. Chem. Intl Ed. 40 (11): 2004-2021 (2001)). In some embodiments, the attachment component can include one functional group or a plurality of functional groups that result in a plurality of covalent bonds with the nanocarrier.

Table 1 provides an additional non-limiting, representative list of functional groups that can be used in the present invention.

TABLE 1
Exemplary Functional Group Pairs for Conjugation Chemistry

| Functional Groups: | Reacts with: |
|---|---|
| Ketone and aldehyde groups | Amino, hydrazido and aminooxy |
| Imide | Amino, hydrazido and aminooxy |
| Cyano | Hydroxy |
| Alkylating agents (such as haloalkyl | Thiol, amino, hydrazido, aminooxy |
| groups and maleimido derivatives) | |
| Carboxyl groups (including activated | Amino, hydroxyl, hydrazido, |
| carboxyl groups) | aminooxy |
| Activated sulfonyl groups (such as | Amino, hydroxyl, hydrazido, |
| sulfonyl chlorides) | aminooxy |
| Sulfhydryl | Sulfhydryl |
| His-tag (such as 6-His tagged peptide | Nickel nitriloacetic acid |
| or protein) | |

In other embodiments, an attachment component can be attached to a nanocarrier by non-covalent interactions that can include but are not limited to affinity interactions, metal coordination, physical adsorption, hydrophobic interactions, van der Waals interactions, hydrogen bonding interactions, magnetic interactions, electrostatic interactions, dipole-dipole interactions, antibody-binding interactions, hybridization interactions between complementary DNA, and the like. In some embodiments, an attachment component can be present in a lipid bilayer portion of a nanocarrier, wherein in certain embodiments the nanocarrier is a liposome. For example, an attachment component can be a lipid that interacts partially or wholly with the hydrophobic and/or hydrophilic regions of the lipid bilayer. In some embodiments, the attachment component can include one group that allows non-covalent interaction with the nanocarrier, but a plurality of groups is also contemplated. For example, a plurality of ionic charges can be used to produce sufficient non-covalent interaction between the attachment component and the nanocarrier. In alternative embodiments, the attachment component can include a plurality of lipids such that the plurality of lipids interacts with a bilayer membrane of a liposome or bilayer or monolayer coated on a nanocarrier. In certain embodiments, surrounding solution conditions can be modified to disrupt non-covalent interactions thereby detaching the attachment component from the nanocarrier.

### Linking Groups

Linking groups are another feature of the targeted delivery compositions of the present invention. One of ordinary skill in the art can appreciate that a variety of linking groups are known in the art and can be found, for example, in the following reference: Hermanson, G. T., Bioconjugate Techniques, 2^{nd} Ed., Academic Press, Inc. (2008). Linking groups of the present invention can be used to provide additional properties to the composition, such as providing spacing between different portions of a conjugate, e.g., A and T. This spacing can be used, for example, to overcome steric hindrance issues caused by the nanocarrier, e.g., when a targeting agent binds to a target. In some embodiments, linking groups can be used to change the physical properties of the targeted delivery composition.

In one group of embodiments, the targeted delivery compositions can include a linking group having the formula: [(EG)(P)]ₙ, wherein the subscript n is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate. In some embodiments, n can be equal to a number sufficient to make the linking group longer than a poly(ethylene glycol) moiety extending from a nanocarrier. In some embodiments, n can be greater than 1. In other embodiments, n can be an integer from 1 to 10, 1 to 20, 1 to 30, or 1 to 40. In yet other embodiments, n can be an integer from 2 to 12, 3 to 12, 4 to 12, 5 to 12, 6 to 12, 7 to 12, 8 to 12, 9 to 12, 10 to 12 and 11 to 12. In yet other embodiments, n can range from 4 to 20, 6 to 20, 8 to 20, 10 to 20, 12 to 20, 14 to 20, 16 to 20, and 18 to 20. In one embodiment, n can be 8. In yet other embodiments, n can be 4, 5, 6, 7, 8, 9, 10, 11 or 12. With respect to EG and P, any combination of both can be used in the linking group. For example, the linking group can be composed of one type of ethylene glycol, such as hexaethylene glycol with only phosphate (HEGp). In other embodiments, different ethylene glycols can be used and combined with any combination of phosphate or thiophosphate. In an exemplary embodiment, the linking group can be tetraethylene glycol-phosphate-hexaethylene glycol-thiophosphate-hexaethylene glycol-phosphate-triethylene glycol-phosphate. One of ordinary skill in the art will appreciate the vast number of combinations available for the linking groups of the present invention.

Illustrated below are a few variations of the described linking groups:

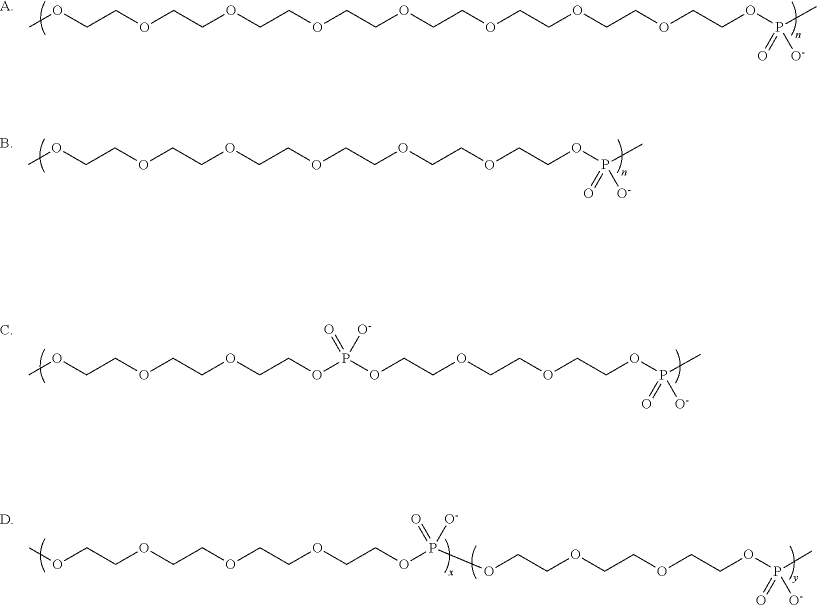

Linking group A shows an octaethylene glycol phosphate. In A, n can be, e.g., between 1 to 20. A can, also, optionally be part of another linking group, or A can be attached to another linking group. Similarly, linking group B shows a hexaethylene glycol phosphate (also described herein as HEGp). B can include a number of repeat units, e.g., n can be between 1 to 20, or preferably about 8. As shown in linking group C, n can equal a specific integer, e.g., n=2, as depicted by an exemplary dimer of triethylene glycol phosphate. Alternatively, linking groups can, e.g., be described using additional subscripts, x and y, such that x+y=n. Linking group D, for example, shows a tetraethylene glycol phosphate linked to a triethylene glycol phosphate. In certain embodiments, the ethylene glycol portions (EG) within the subscripted brackets of x and y can be independently selected from a group consisting of triethylene glycol, tetraethylene glycol, pentaethylene glycol, hexaethylene glycol, heptaethylene glycol, and octaethylene glycol.

### Therapeutic Agents

The nanocarriers used in the targeted therapeutic or diagnostic delivery compositions of the present invention include a therapeutic agent, diagnostic agent, or a combination thereof. The therapeutic agent and/or diagnostic agent can be present anywhere in, on, or around the nanocarrier. In some embodiments, the therapeutic agent and/or diagnostic agent can be embedded in, encapsulated in, or tethered to the nanocarrier. In certain embodiments, the nanocarrier is a liposome and the diagnostic and/or therapeutic agent is encapsulated in the liposome.

A therapeutic agent used in the present invention can include any agent directed to treat a condition in a subject. In general, any therapeutic agent known in the art can be used, including without limitation agents listed in the United States Pharmacopeia (U.S.P.), Goodman and Gilman's The Pharmacological Basis of Therapeutics, 10^{th} Ed., McGraw Hill, 2001; Katzung, Ed., Basic and Clinical Pharmacology, McGraw-Hill/Appleton & Lange, 8^{th} ed., Sep. 21, 2000; Physician's Desk Reference (Thomson Publishing; and/or The Merck Manual of Diagnosis and Therapy, 18^{th} ed., 2006, Beers and Berkow, Eds., Merck Publishing Group; or, in the case of animals, The Merck Veterinary Manual, 9^{th} ed., Kahn Ed., Merck Publishing Group, 2005; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, shRNA and/or shRNA agents.

In some embodiments, a therapeutic agent can include an anticancer agent or cytotoxic agent including but not limited to avastin, doxorubicin, cisplatin, oxaliplatin, carboplatin, 5-fluorouracil, gemcitibine or taxanes, such as paclitaxel and docetaxel. Additional anti-cancer agents can include but are not limited to 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-aletheine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, alelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, foternustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lotnbricine, lometrexol, lometrexol sodium, lomustine, lonidamine, losoxantrone, losoxantrone hydrochloride, lovastatin, loxoribine, lurtotecan, lutetium texaphyrin, lisofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, membrane, mercaptopurine, meterelin, methioninate, methotrexate, methotrexate sodium, metoclopramide, metoprine, meturedepa, microalgal protein kinase C inhibitors, MiF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/mycobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytrioi, panomifene, parabactin, pazelliptine, pegaspargase, peldesine, peliornycin, pentamustine, pentosan polysulfate sodium, pentostatin, pentrazole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J12, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl; safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spiramycin I), spirogermanium hydrochloride, spiromustine, spiroplatin, splenopentin, spongistatin 1, squalamine, stem cell inhibitor, stem-cell division inhibitors, stipiamide, streptonigrin, streptozocin, strornelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

In some embodiments, the therapeutic agents can be part of cocktail of agents that includes administering two or more therapeutic agents. For example, a liposome having both cisplatin and oxaliplatin can be administered. In addition, the therapeutic agents can be delivered before, after, or with immune stimulatory adjuvants, such as aluminum gel or salt adjuvants (e.g., aluminium phosphate or aluminum hydroxide), calcium phosphate, endotoxins, toll-like receptor adjuvants and the like.

Therapeutic agents of the present invention can also include radionuclides for use in therapeutic applications. For example, emitters of Auger electrons, such as ¹¹¹In, can be combined with a chelate, such as diethylenetriaminepentaacetic acid (DTPA) or 1,4,7,10-tetraazacyclododecane-1,4,7,10-tetraacetic acid (DOTA), and included in a targeted delivery composition, such as a liposome, to be used for treatment. Other suitable radionuclide and/or radionuclide-chelate combinations can include but are not limited to beta radionuclides (¹⁷⁷Lu, ¹⁵³Sm, ^{88/90}Y) with DOTA, ⁶⁴Cu-TETA, ^{188/186}Re(CO)₃-IDA; ^{188/186}Rc(CO)triamines (cyclic or linear), ^{188/186}Re(CO)₃-Enpy2, and ^{188/186}Re(CO)₃-DTPA.

As described above, the therapeutic agents used in the present invention can be associated with the nanocarrier in a variety of ways, such as being embedded in, encapsulated in, or tethered to the nanocarrier. Loading of the therapeutic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006). In a group of embodiments, one or more therapeutic agents can be loaded into liposomes. Loading of liposomes can be carried out, for example, in an active or passive manner. For example, a therapeutic agent can be included during the self-assembly process of the liposomes in a solution, such that the therapeutic agent is encapsulated within the liposome. In certain embodiments, the therapeutic agent may also be embedded in the liposome bilayer or within multiple layers of multilamellar liposome. In alternative embodiments, the therapeutic agent can be actively loaded into liposomes. For example, the liposomes can be exposed to conditions, such as electroporation, in which the bilayer membrane is made permeable to a solution containing therapeutic agent thereby allowing for the therapeutic agent to enter into the internal volume of the liposomes.

### Diagnostic Agents

A diagnostic agent used in the present invention can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, chogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8,11-tetraazacyclotetradec-1-yl)methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ⁸⁸⁻⁹⁰Y, ^{62/64/67}Cu, or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging. 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenapyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to Gadopentetic acid, Gadoteric acid, Gadodiamide, Gadolinium, Gadoteridol, Mangafodipir, Gadoversetamide, Ferric ammonium citrate, Gadobenic acid, Gadobutrol, or Gadoxetic acid. Superparamagnetic agents can include but are not limited to superparamagnetic iron oxide and Ferristene. In certain embodiments, the diagnostic agents can include x-ray contrast agents as provided, for example, in the following references: ELS Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexyl, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexyl, iopentol, ioversol, iobitridol, iodixanol, iotrolan and iosimenol.

Similar to therapeutic agents described above, the diagnostic agents can be associated with the nanocarrier in a variety of ways, including for example being embedded in, encapsulated in, or tethered to the nanocarrier. Similarly, loading of the diagnostic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006).

### Targeting Agents

The targeted delivery compositions of the present invention also include T, a targeting agent. Generally, the targeting agents of the present invention can associate with any target of interest, such as a target associated with an organ, tissues, cell, extracellular matrix, or intracellular region. In certain embodiments, a target can be associated with a particular disease state, such as a cancerous condition. Alternatively, a targeting component can target one or more particular types of cells that can, for example, have a target that indicates a particular disease and/or particular state of a cell, tissue, and/or subject. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell. In certain embodiments, suitable targets can include mucins such as MUC-1 and MUC-4, growth factor receptors such as EGFR, Claudin 4, nucleolar phosphoproteins such as nucleolin, chemokine receptors such as CCR7, receptors such as somatostatin receptor 4, Erb-B2 (erythroblastic leukaemia oncogene homologue 2) receptor, CD44 receptor, and VEGF receptor-2 kinase.

In certain embodiments, a targeting agent can include a small molecule mimic of a target ligand (e.g., a peptide mimetic ligand), a target ligand (e.g., an RGD peptide containing peptide or folate amide), or an antibody or antibody fragment specific for a particular target. In some embodiments, a targeting agent can further include folic acid derivatives, B-12 derivatives, integrin RGD peptides, NGR derivatives, somatostatin derivatives or peptides that bind to the somatostatin receptor, e.g., octreotide and octreotate, and the like.

The targeting agents of the present invention can also include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)). In the present invention, suitable aptamers can be linear or cyclized and can include oligonucleotides having less than about 150 bases (i.e., less than about 150 mer). Aptamers can range in length from about 100 to about 150 bases or from about 80 to about 120 bases. In certain embodiments, the aptamers can range from about 12 to 40 about bases, from about 12 to about 25 bases, from about 18 to about 30 bases, or from about 15 to about 50 bases. The aptamers can be developed for use with a suitable target that is present or is expressed at the disease state, and includes, but is not limited to, the target sites noted herein.

### B. Individual Components of the Targeted Delivery Compositions Including a Nanocarrier

In another aspect, the present invention provides individual components of the targeted delivery compositions disclosed herein. In particular, the present invention includes a conjugate having the formula: A-[(EG)(P)]ₙ-T; wherein, A is an attachment component; [(EG)(P)]ₙ is a linking group, wherein the subscript n is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate; and, T is a targeting agent.

It will be appreciated by one of ordinary skill in the art that components of the targeted delivery compositions similarly include each of the specific embodiments described above.

C. Targeted Delivery Compositions Including A Diagnostic and/or Therapeutic Agent Directly Attached to a Linking Group

In yet another aspect, the present invention provides targeted delivery compositions wherein a diagnostic and/or therapeutic agent is directly attached to a linking group. In one embodiment, the targeted delivery compositions of the present invention include a conjugate having the formula: (DT)-[(EG)(P)]ₘ-T; wherein, DT is a diagnostic agent, a therapeutic agent, or a combination thereof; [(EG)(P)]ₘ is a linking group, wherein the subscript m is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate; and, T is a targeting agent.

In one group of embodiments, the targeted delivery compositions can include a diagnostic and/or therapeutic component directly attached to a linking group having the formula: [(EG)(P)]ₘ, wherein the subscript m is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate. As compared to the targeted delivery compositions including a nanocarrier, the number of ethylene glycol groups in the linking group can be less because, for some instances, steric or other considerations may not exist with the compositions not including a nanocarrier. In some embodiments, m can be greater than 1. In other embodiments, m can be an integer from 1 to 10, 1 to 20, or Ito 30. In yet other embodiments, m can be an integer from 2 to 12, 3 to 12, 4 to 12, 5 to 12, 6 to 12, 7 to 12, 8 to 12, 9 to 12, 10 to 12 and 11 to 12. In yet other embodiments, m can range from 4 to 20, 6 to 20, 8 to 20, 10 to 20, 12 to 20, 14 to 20, 16 to 20, and 18 to 20. In one embodiment, m can be 8. In yet other embodiments, m can be 4, 5, 6, 7, 8, 9, 10, 11 or 12. With respect to EG and P, any combination of both can be used in the linking group. For example, the linking group can be composed of one type of ethylene glycol, such as hexaethylene glycol along with only phosphate (HEGp). In other embodiments, different ethylene glycols can be used and combined with any combination of phosphate or thiophosphate. In an exemplary embodiment, the linking group can be tetraethylene glycol-phosphate-hexaethylene glycol-thiophosphate-hexaethylene glycol-phosphate-triethylene glycol-phosphate. In yet other embodiments, another linking group or functional group can optionally be used to attach [(EG)(P)]ₘ to DT. For example, depending on the therapeutic and/or diagnostic agent, one of ordinary skill in the art may employ any of the functional groups or bifunctional linking groups described above to attach [(EG)(P)]ₘ to DT. In certain embodiments, both [(EG)(P)]ₘ and DT may terminate with a hydroxy group. An exemplary linking chemistry for these embodiments can include, but is not limited to, α-halo ester linking chemistry, such as linkages formed using ethyl 2-bromoacetate. One of ordinary skill in the art will appreciate that a number of combinations are available for the linking groups of the present invention.

In general, it will be appreciated by one of ordinary skill in the art that the selected embodiments of the targeted delivery compositions including a nanocarrier as described above can be similarly applied to the embodiments disclosed herein for targeted delivery compositions wherein a diagnostic and/or therapeutic agent is directly attached to a linking group. Methods for attaching the diagnostic and/or therapeutic agents to the linking groups are well known in the art and typically include covalent attachments that are described in more detail above. DT can include any of the therapeutic and/or diagnostic agents that are described above and directly provides the therapeutic and/or diagnostic agent to a subject without the need for a nanocarrier.

### IV. Methods of Preparing Targeted Delivery Compositions and Components

### A. Targeted Delivery Compositions Including a Nanocarrier

The targeted delivery compositions of the present invention can be produced in a variety of ways. In one aspect, targeted delivery compositions of the present invention can be prepared using a method of preparing a targeted delivery composition, comprising attaching a nanocarrier including a therapeutic or diagnostic agent to a conjugate having the formula: A-[(EG)(P)]ₙ-T; wherein, A is an attachment component for attaching said conjugate to said nanocarrier; [(EG)(P)]ₙ is a linking group, wherein the subscript n is an integer from 1 to about 40; and each EG is independently selected from a group consisting of triethylene glycol, tetraethylene glycol, pentaethylene glycol, hexaethylene glycol, heptaethylene glycol, and octaethylene glycol; P is independently selected from a group consisting of phosphate and thiophosphate; and, T is a targeting agent.

### Nanocarriers

Nanocarriers can be produced by a variety of ways generally known in the art and methods of making such nanocarriers can depend on the particular nanocarrier desired. Any measuring technique available in the art can be used to determine properties of the targeted delivery compositions and nanocarriers. For example, techniques such as dynamic light scattering, x-ray photoelectron microscopy, powder x-ray diffraction, scanning electron microscopy (SEM), transmission electron microscopy (TEM), and atomic force microscopy (AFM) can be used to determine average size and dispersity of the nanocarriers and/or targeted delivery compositions.

Liposomes used in the targeted delivery compositions of the present invention can be made using a variety of techniques generally well known in the art. (See, e.g., Williams, A. P., Liposomes: A Practical Approach, 2^{nd} Edition, Oxford Univ. Press (2003); Lasic, D. D., Liposomes in Gene Delivery, CRC Press LLC (1997)). For example, liposomes can be produced by but are not limited to techniques such as extrusion, agitation, sonication, reverse phase evaporation, self-assembly in aqueous solution, electrode-based formation techniques, microfluidic directed formation techniques, and the like. In certain embodiments, methods can be used to produce liposomes that are multilamellar and/or unilamellar, which can include large unilamellar vesicles (LUV) and/or small unilamellar vesicles (SUV). Similar to self-assembly of liposomes in solution, micelles can be produced using techniques generally well known in the art, such that amphiphilic molecules will form micelles when dissolved in solution conditions sufficient to form micelles. Lipid-coated bubbles and lipoproteins can also be constructed using methods known in the art (See, e.g., Farook, U., J. R. Soc. Interface, 6(32): 271-277 (2009); Lacko et al., Lipoprotein Nanocarriers as Delivery Vehicles for Anti-Cancer Agents in Nanotechnology for Cancer Therapy, CRC Press (2007)).

Methods of making polymeric nanocarriers that can be used in the present invention are generally well known in the art (See, e.g., Sigmund, W. et al., Eds., Particulate Systems in Nano- and Biotechnologies, CRC Press LLC (2009); Karnik et al., Nano Lett., 8(9): 2906-2912 (2008)). For example, block copolymers can be made using synthetic methods known in the art such that the block copolymers can self-assemble in a solution to form polymerosomes and/or block copolymer micelles. Niosomes are known in the art and can be made using a variety of techniques and compositions (Baillie A. J. et al, J. Pharm. Pharmacol., 38:502-505 (1988)). Magnetic and/or metallic particles can be constructed using any method known in the art, such as co-precipitation, thermal decomposition, and microemulsion. (See also Nagarajan, R. & Hatton, T. A., Eds., Nanocarriers Synthesis, Stabilization, Passivation, and Functionalization, Oxford Univ. Press (2008)). Gold particles and their derivatives can be made using a variety of techniques generally known in the art, such as the Turkevich method, Brust method, Perraut Method or sonolysis (See also, Grzelczak et al., Chem. Soc. Rev., 37: 1783-1791 (2008)). In some embodiments, the attachment component can be attached through sulfur-gold tethering chemistry. Quantum dots or semiconductor nanocrystals can be synthesized using any method known in the art, such as colloidal synthesis techniques. Generally, quantum dots can be composed of a variety of materials, such as semiconductor materials including cadmium selenide, cadmium sulfide, indium arsenide, indium phosphide, and the like.

### Conjugates for Attaching to a Nanocarrier

The conjugates having the formula A-[(EG)(P)]ₙ-T, as described further herein, can be manufactured using a variety of techniques. In some embodiments, the entire conjugate can be synthesized in oligonucleotide synthesizers well known in the art. Using phosphoramidite synthesis, for example, nucleotide sequences including standard bases (e.g., dG, dT, dA, or dC) can be synthesized using standard DNA synthesis cycles. In certain embodiments, incorporation of [(EG)(P)]ₙ, such as (HEGp)ₙ, can be performed using modified synthesis cycles for more effective incorporation. In particular, increased amidite equivalents and extended wash cycles can incorporate multiple [(EG)(P)] units as linking groups in the conjugates of the present invention. In certain embodiments, an attachment component, such as cholesterol or a cholesterol derivative (e.g., cholesterol-tetraethylene glycol) can then be added using standard or modified synthesis cycles, which can include doubling the coupling recycle step to insure effective incorporation. In certain embodiments, the conjugates can be synthesized using solid phase approaches, such as silica-based or polystyrene-based supports.

In other embodiments, the [(EG)(P)]ₙ linking group can be attached to an attachment component, such as a cholesterol derivative (cholesterol-tetraethylene glycol), using conventional chemistry known in the art. The [(EG)(P)]ₙ linking group can be synthesized using the methods described above. Next, the linking group and the attachment component can be mixed and reacted under conditions sufficient to form a portion of the conjugate, A-[(EG)(P)]ₙ. Subsequently, a targeting agent, e.g., an aptamer, can be attached to the other end of the [(EG)(P)]ₙ linking group. Alternatively, the targeting agent can be attached to the [(EG)(P)]ₙ linking group first, followed by the attachment component. As will be appreciated by one of ordinary skill in the art, targeting agents of the present invention can be attached to the [(EG)(P)]ₙ linking group by a variety of ways that can depend on the characteristics of the targeting agent. For example, reaction syntheses can be different if the targeting agent is composed of peptides, nucleotides, carbohydrates, and the like.

In certain embodiments, the targeting agent can include an aptamer. Aptamers for a particular target can be identified using techniques known in the art, such as but not limited to, in vitro selection processes, such as SELEX (systematic evolution of ligands by exponential enrichment), or MonoLex™ technology (single round aptamer isolation procedure for AptaRes AG), in vivo selection processes, or combinations thereof (See e.g., Ellington, A. D. & Szostak, J. W., Nature 346(6287): 818-22; Bock et al., Nature 355(6360): 564-6 (1992)). In some embodiments, the above mentioned methods can be used to identify particular DNA or RNA sequences that can be used to bind a particular target site of interest, as disclosed herein. Once a sequence of a particular aptamer has been identified, the aptamer can be constructed in a variety of ways known in the art, such as phosphoramidite synthesis. For peptide aptamers, a variety of identification and manufacturing techniques can be used (See e.g., Colas, P., J. Biol. 7:2 (2008); Woodman, R. et al., J. Mol. Biol. 352(5): 1118-33 (2005).

Similar to the reaction sequence described above, aptamers can be attached to the [(EG)(P)]ₙ linking group by a variety of ways. For example, the [(EG)(P)]ₙ linking group can be reacted with a 3′ or 5′ end of the aptamer. In some embodiments, the aptamer can be attached to [(EG)(P)]ₙ linking group after the attachment component has been reacted with the other end of the [(EG)(P)]ₙ linking group. In other embodiments, the aptamer can be attached to the [(EG)(P)]ₙ linking group first and then followed by attachment of the attachment component (e.g., cholesterol-tetraethylene glycol). In alternative embodiments, the aptamer can be synthesized sequentially by adding one nucleic acid at a time to the end of the [(EG)(P)]ₙ linking group. In yet other embodiments, the attachment component and the targeting agent, e.g., the aptamer, can be placed in the same reaction vessel to form the conjugate all in one step.

B. Targeted Delivery Compositions Including A Diagnostic and/or Therapeutic Agent Directly Attached to a Linking Group

The conjugates having the formula DT-[(EG)(P)]ₘ-T can be prepared using methods generally well known in the art. In certain embodiments, a chelator can be attached to a [(EG)(P)]ₘ linking group and then a targeting agent can be attached to the other end of the [(EG)(P)]ₘ linking group. A radioisotope can then be complexed with the chelator. The present invention, however, contemplates several orders of steps for making the conjugates. In some embodiments, certain steps can be reversed. For example, a chelator can be combined with a radioisotope to form the diagnostic component that can then be further reacted using conventional chemistry with a [(EG)(P)]ₘ linking group. The targeting agent, e.g., an aptamer, can then be attached to the other end of the [(EG)(P)]ₘ linking group as described herein. In yet another aspect, a therapeutic agent can be attached to a [(EG)(P)]ₘ linking group and the targeting agent, e.g., an aptamer, can be attached to the opposite end of the linking group, as described herein. One of ordinary skill in the art will appreciate that the diagnostic and/or therapeutic components can be constructed in several different ways other than the examples provided above. In addition, making the diagnostic or therapeutic components can depend on the particular diagnostic and/or therapeutic agent being used.

### V. Methods of Administering Targeted Delivery Compositions

As described herein, the targeted delivery compositions and methods of the present invention can be used for treating and/or diagnosing any disease, disorder, and/or condition associated with a subject. In one embodiment, the methods of the present invention include a method for treating or diagnosing a cancerous condition in a subject, comprising administering to the subject a targeted delivery composition of the present invention that includes a nanocarrier, wherein the therapeutic or diagnostic agent is sufficient to treat or diagnose the condition. In certain embodiments, the cancerous condition can include cancers that sufficiently express (e.g., on the cell surface or in the vasculature) a receptor that is being targeted by a targeting agent of a targeted delivery composition of the present invention.

In another embodiment, the methods of the present invention include a method of determining the suitability of a subject for a targeted therapeutic treatment, comprising administering to the subject a targeted delivery composition that includes a nanocarrier, wherein the nanocarrier comprises a diagnostic agent, and imaging the subject to detect the diagnostic agent.

In yet another embodiment, the methods of the present invention include a method for treating or diagnosing a cancerous condition in a subject, comprising administering to the subject a targeted delivery composition of the present invention including a diagnostic and/or therapeutic agent directly attached to a [(EG)(P)]ₘ linking group, wherein the therapeutic or diagnostic agent is sufficient to treat or diagnose the condition.

In yet another embodiment, the methods of the present invention include a method of determining the suitability of a subject for a targeted therapeutic treatment, comprising administering to said subject a targeted delivery composition of the present invention comprising a diagnostic agent directly attached to a [(EG)(P)]ₘ linking group, and imaging said subject to detect the diagnostic agent.

### Administration

In some embodiments, the present invention can include a targeted delivery composition and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized targeted delivery compositions.

The targeted delivery composition of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged targeted delivery composition with a suppository base. Suitable suppository bases include natural or synthetic triglycerides or paraffin hydrocarbons. In addition, it is also possible to use gelatin rectal capsules which contain a combination of the targeted delivery composition of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of targeted delivery compositions can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a targeted delivery composition. The unit dosage form can be a packaged preparation, the package containing discrete quantities of preparation. The composition can, if desired, also contain other compatible therapeutic agents.

In therapeutic use for the treatment of cancer, the targeted delivery compositions including a therapeutic and/or diagnostic agent utilized in the pharmaceutical compositions of the present invention can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the targeted delivery composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted delivery composition in a particular patient. Determination of the proper dosage for a particular situation is within the skill of the practitioner. Generally, treatment is initiated with smaller dosages which are less than the optimum dose of the targeted delivery composition. Thereafter, the dosage is increased by small increments until the optimum effect under circumstances is reached. For convenience, the total daily dosage may be divided and administered in portions during the day, if desired.

In some embodiments, the targeted delivery compositions of the present invention may be used to diagnose a disease, disorder, and/or condition. In some embodiments, the targeted delivery compositions can be used to diagnose a cancerous condition in a subject, such as lung cancer, breast cancer, pancreatic cancer, prostate cancer, cervical cancer, ovarian cancer, colon cancer, liver cancer, esophageal cancer, and the like. In some embodiments, methods of diagnosing a disease state may involve the use of the targeted delivery compositions to physically detect and/or locate a tumor within the body of a subject. For example, tumors can be related to cancers that sufficiently express (e.g., on the cell surface or in the vasculature) a receptor that is being targeted by a targeting agent of a targeted delivery composition of the present invention. In some embodiments, the targeted delivery compositions can also be used to diagnose diseases other than cancer, such as proliferative diseases, cardiovascular diseases, gastrointestinal diseases, genitourinary disease, neurological diseases, musculoskeletal diseases, hematological diseases, inflammatory diseases, autoimmune diseases, rheumatoid arthritis and the like.

As disclosed herein, the targeted delivery compositions of the invention can include a diagnostic agent that has intrinsically detectable properties. In detecting the diagnostic agent in a subject, the targeted delivery compositions, or a population of particles with a portion being targeted delivery compositions, can be administered to a subject. The subject can then be imaged using a technique for imaging the diagnostic agent, such as single photon emission computed tomography (SPECT), magnetic resonance imaging (MRI), optical imaging, positron emission tomography (PET), computed tomography (CT), x-ray imaging, gamma ray imaging, and the like. Any of the imaging techniques described herein may be used in combination with other imaging techniques. In some embodiments, the incorporation of a radioisotope for imaging in a particle allows in vivo tracking of the targeted delivery compositions in a subject. For example, the biodistribution and/or elimination of the targeted delivery compositions can be measured and optionally be used to alter the treatment of patient. For example, more or less of the targeted delivery compositions may be needed to optimize treatment and/or diagnosis of the patient.

### Targeted Delivery

In certain embodiments, the targeted delivery compositions of the present invention can be delivered to a subject to release a therapeutic or diagnostic agent in a targeted manner. For example, a targeted delivery composition can be delivered to a target in a subject and then a therapeutic agent embedded in, encapsulated in, or tethered to the targeted delivery composition, such as to the nanocarrier, can be delivered based on solution conditions in vicinity of the target. Solution conditions, such as pH, salt concentration, and the like, may trigger release over a short or long period of time of the therapeutic agent to the area in the vicinity of the target. Alternatively, an enzyme can cleave the therapeutic or diagnostic agent from the targeted delivery composition to initiate release. In some embodiments, the targeted delivery compositions can be delivered to the internal regions of a cell by endocytosis and possibly later degraded in an internal compartment of the cell, such as a lysosome. One of ordinary skill will appreciate that targeted delivery of a therapeutic or diagnostic agent can be carried out using a variety of methods generally known in the art.

### Kits

The present invention also provides kits for administering the targeted delivery compositions to a subject for treating and/or diagnosing a disease state. Such kits typically include two or more components necessary for treating and/or diagnosing the disease state, such as a cancerous condition. Components can include targeted delivery compositions of the present invention, reagents, containers and/or equipment. In some embodiments, a container within a kit may contain a targeted delivery composition including a radiopharmaceutical that is radiolabeled before use. The kits can further include any of the reaction components or buffers necessary for administering the targeted delivery compositions. Moreover, the targeted delivery compositions can be in lyophilized form and then reconstituted prior to administration.

In certain embodiments, the kits of the present invention can include packaging assemblies that can include one or more components used for treating and/or diagnosing the disease state of a patient. For example, a packaging assembly may include a container that houses at least one of the targeted delivery compositions as described herein. A separate container may include other excipients or agents that can be mixed with the targeted delivery compositions prior to administration to a patient. In some embodiments, a physician may select and match certain components and/or packaging assemblies depending on the treatment or diagnosis needed for a particular patient.

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Examples

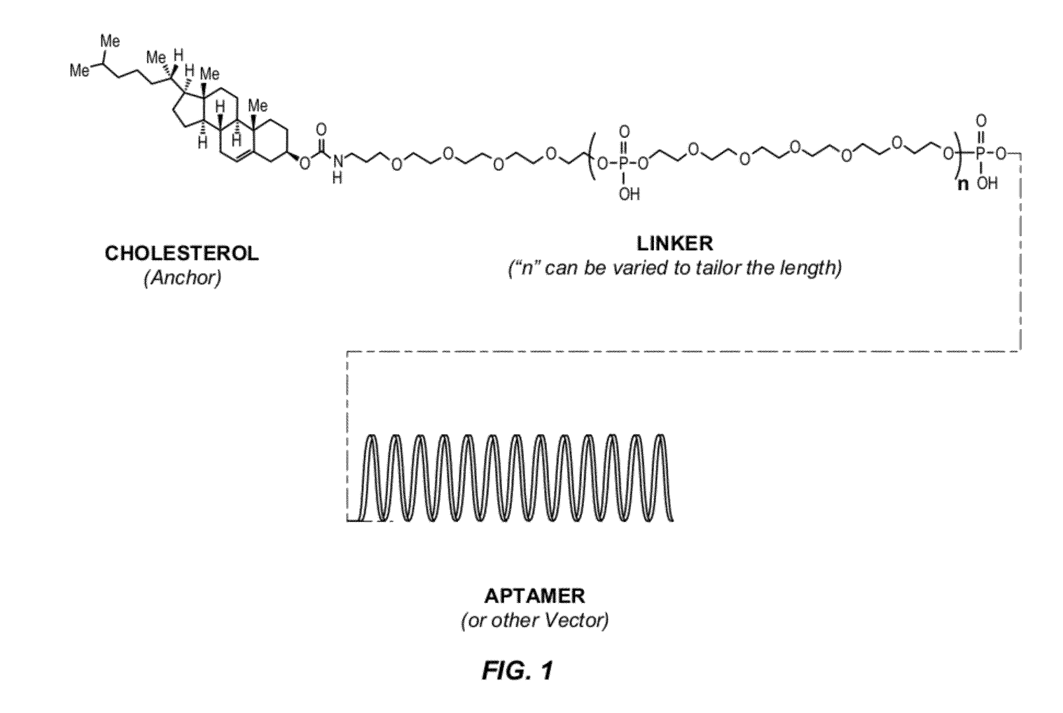

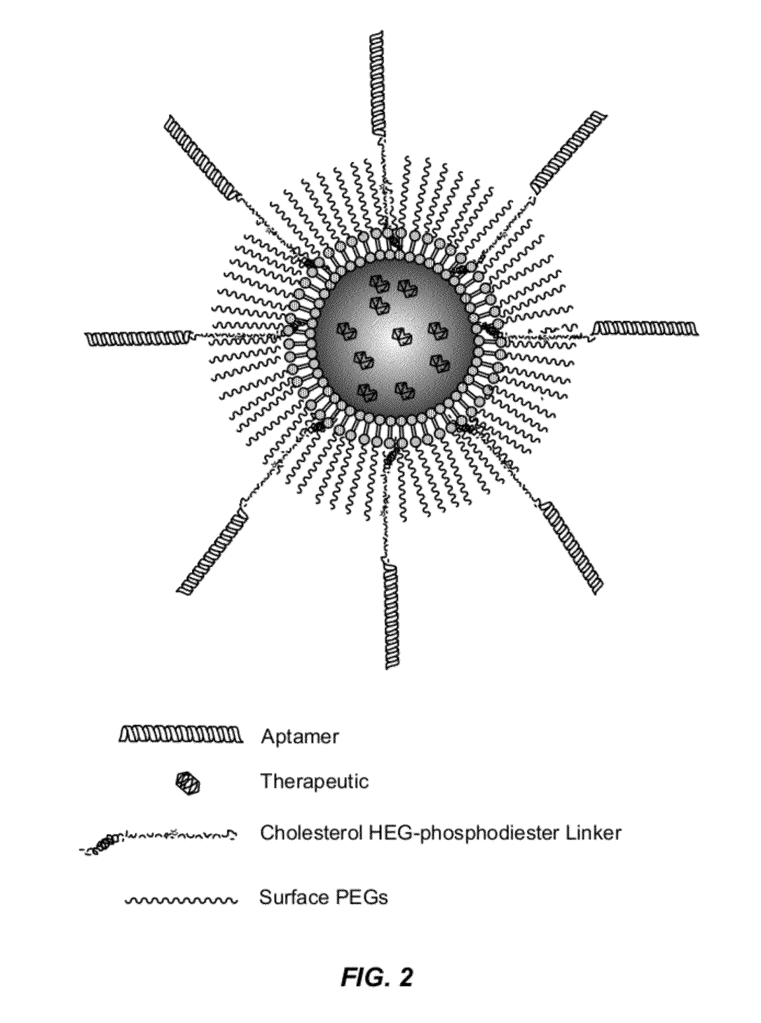

FIG. 1 provides a generic illustration of an aptamer-(HEGp)ₙ-cholesterol conjugate, as described herein. The cholesterol can function to anchor the conjugate to the hydrophobic region of a nanocarrier. In the specific case of liposomes, the cholesterol can be anchored within the hydrophobic region of the phospholipid bilayer membrane. Cholesterol is a common additive in liposome formulations for fluidizing the gel state and allowing lateral diffusion of components within the bilayer. The linker is synthesized from individual monomers of hexaethyleneglycol (HEG) via solid-phase phosphoramidite chemistry. The phosphoramidite approach places a phosphate group after every HEG unit in the linker chain. Accordingly, the number of HEGp monomers in the chain can be increased or decreased for optimization of the distance between the targeting aptamer and the nanocarrier and any/or surface PEGs. FIG. 2 depicts an exemplary image of a targeted therapeutic liposome incorporating the exemplary aptamer-(HEGp)ₙ-cholesterol conjugate.

### A. Synthesis of an AS1411-(HEGp)₈-Cholesterol Conjugate

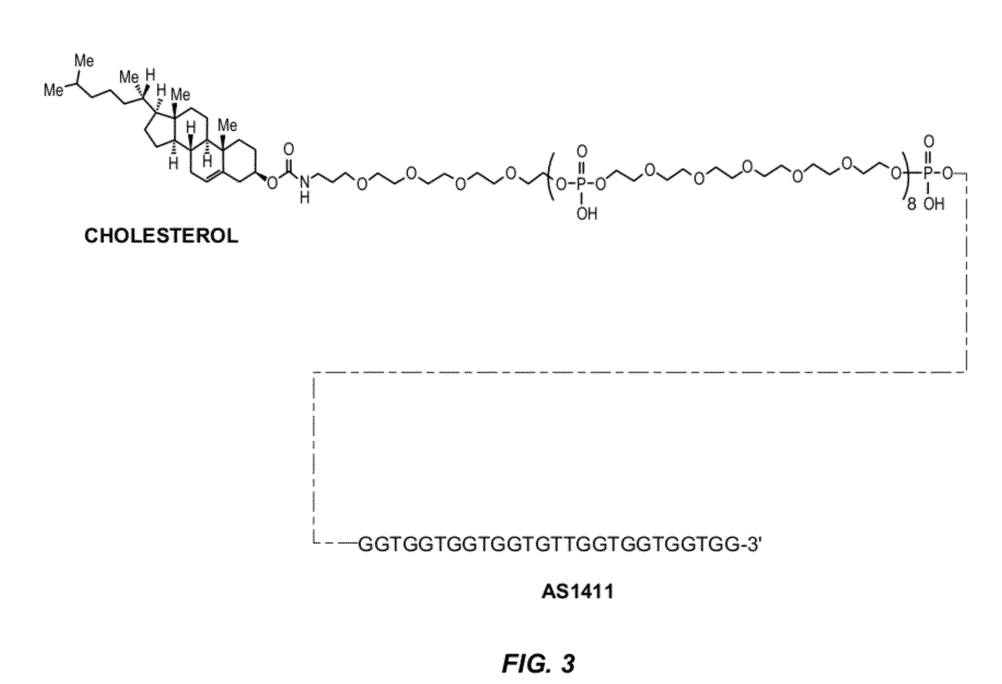

In an exemplary embodiment of the invention, the specific conjugate in FIG. 3 was prepared. This example conjugate employs the known aptamer AS1411, which binds to nucleolin. Nucleolin has been shown to be present at elevated levels in the cytoplasm and on the surface of cancer cells. The sequence of AS 1411 is 5′-GGTGGTGGTGGTGTTGGTGGTGGTGG-3′.

The entire conjugate was assembled via automated synthesis on an ÄKTA Oligopilot Plus oligonucleotide synthesizer (GE Healthcare). The synthesis was performed using the Custom Primer Support 200 dG 80s polystyrene-based resin (GE Healthcare) at a synthesis scale of 97 μmol. All phosphoramidites (dG, dT, cholesterol, and HEG) were purchased from ChemGenes, Inc. Standard DNA synthesis cycles were used to build up the aptamer sequence. For effective incorporation of multiple units of the HEGp, modified synthesis cycles employing increased amidite equivalents and extended wash cycles were used. For addition of the cholesterol at the 5′-end of the conjugate, the coupling recycle step was doubled in order to insure effective incorporation. Coupling efficiencies for the standard nucleotides were >98% at each step based on trityl monitoring at 350 nm. The coupling efficiencies of the HEGp units ranged from 94-96%.

### B. Post-Synthesis Workup

Upon completion of the synthesis, the resin was dried under vacuum for 90 minutes and transferred into a 100 mL pressure vessel. The conjugate was then deprotected and cleaved from the support by treating with concentrated ammonium hydroxide at 55° C. for 5 hours inside the sealed pressure vessel. After deprotection, the suspension was cooled to room temperature, and the released aptamer conjugate was separated from the spent solid support by vacuum filtration. The support was further rinsed with 2×40 mL 50% ethanol, followed by 2×40 mL dH₂O. The sample was then diluted to 200 mL total volume with water, and the crude material analyzed by UPLC & LC/MS. HPLC showed several fast-eluting failure sequences, with one major late-eluting peak as expected for the full-length product containing the cholesterol. LC/MS of this major late-eluting peak was consistent with the mass of the desired product.

### C. Purification by High Performance Liquid Chromatography (HPLC)

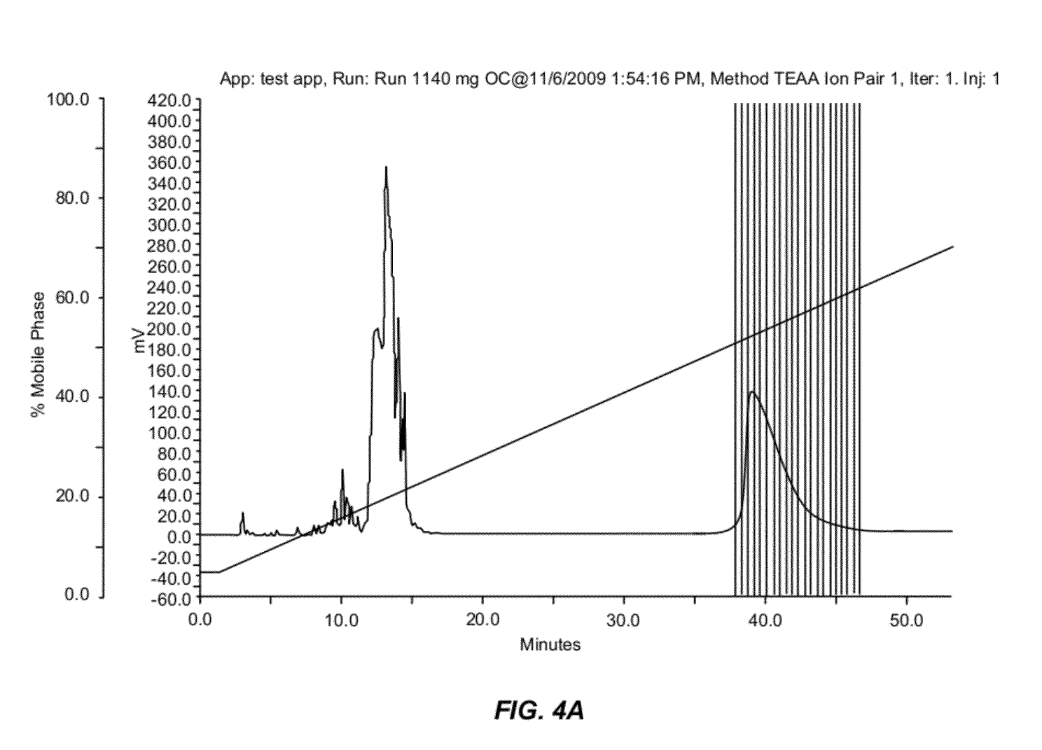

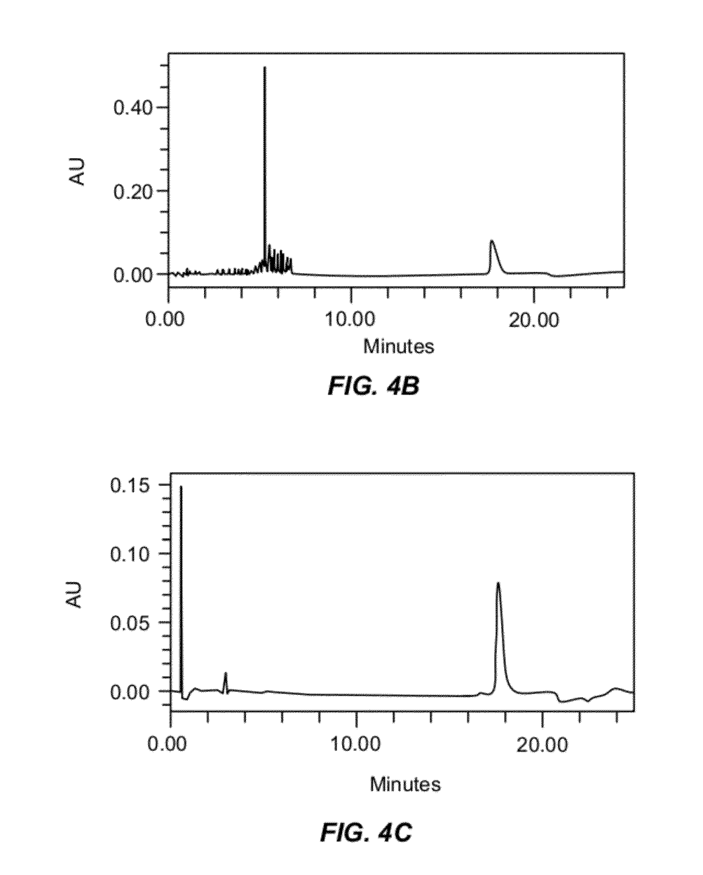

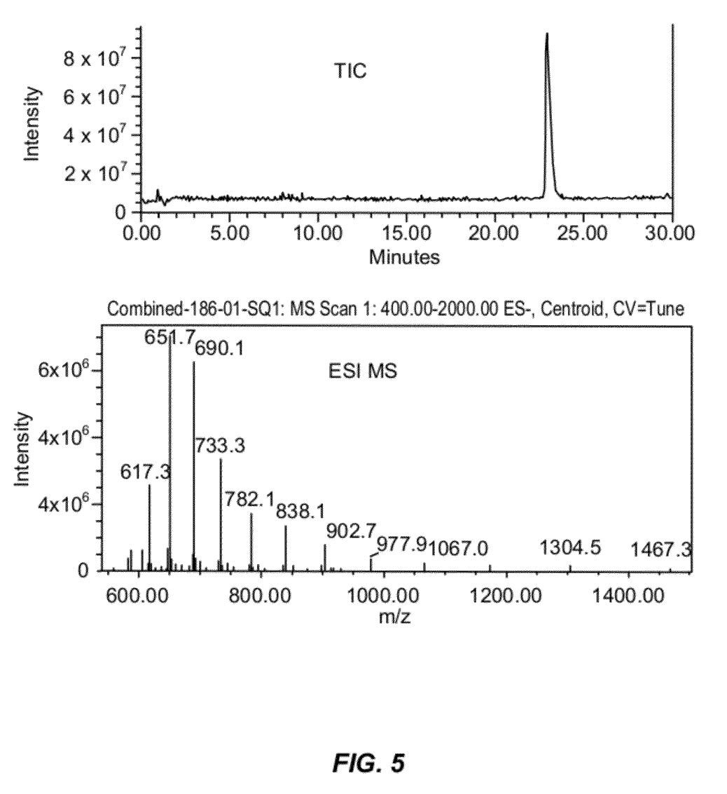

The cleavage solution containing the conjugate and failure-sequence impurities was evaporated to dryness (rotary evaporation, 45° C., 15 mm Hg) and further dried under moderate vacuum 1 hour. The residue so obtained was dissolved in mobile phase A (see below) at an approximate concentration of 40 mg/mL. The sample was purified by injection onto a reversed phase HPLC column (125 mg on-column, Phenomenex Clarity Oligo RP Axia, 30×250 mm), followed by elution at ambient temperature at 45 mL/min using a linear gradient under ion pairing conditions (5-80% B/60 minutes; A=100 mM triethylammonium acetate, pH 8; B=acetonitrile), while monitoring at 260 nm. The desired product eluted at 38-43 minutes, as shown in the trace in FIG. 4A; failure sequences and most other impurities eluted before 15 minutes. The product was collected at regular intervals across the product peak as a series of 20 mL fractions. The fractions were analyzed by Ultra Performance Liquid Chromatography (HPLC), by injection onto a reversed phase HPLC column (Waters Acquity OST C18, 1.7 μm, 2.1×50 mm) held at 60° C., followed by elution at 0.25 mL/min using a linear gradient under ion pairing conditions (30% B-70% B/10 minutes; A=1% v/v 1,1,1,3,3,3-hexafluoroisopropanol, 0.1% diisopropylethylamine, 10 μM EDTA; B=0.1% v/v 1,1,1,3,3,3-hexafluoroisopropanol, 0.05% diisopropylethylamine, 10 μM EDTA, 50% v/v acetonitrile), while monitoring at 260 nm. The desired product eluted at 6.5-7 minutes, as shown in the trace in FIG. 4B (crude product) and FIG. 4C (purified product). The m/z (electrospray ionization, negative ion mode) of the main peak in the chromatogram was consistent with the proposed structure. (Experimental Exact Mass: 11747.9 Da); Calculated: 11746.8 Da). The total ion current and mass spectrum of the product, indicating negatively charged ions (charges: −19 to −9) are shown in FIG. 5.

What is claimed is:

1. A targeted delivery composition, comprising:
(a) a nanocarrier including a therapeutic or diagnostic agent or a combination thereof; and
(b) a conjugate having the formula:
A-[(EG)(P)]ₙ-T;
wherein,
A is an attachment component for attaching said conjugate to said nanocarrier;
[(EG)(P)]ₙ is a linking group, wherein the subscript n is an integer from 1 to about 40; and
each EG is independently selected from a group consisting of triethylene glycol, tetraethylene glycol, pentaethylene glycol, hexaethylene glycol, heptaethylene glycol, and octaethylene glycol;
P is independently selected from a group consisting of phosphate and thiophosphate; and,
T is a targeting agent.

2. The targeted delivery composition of claim 1, wherein said nanocarrier is selected from the group consisting of a liposome, a micelle, a lipoprotein, a lipid-coated bubble, a block copolymer micelle, a polymersome, a noisome, an iron oxide particle, a gold particle, a silica particle, a dendrimer, and a quantum dot.

3. The targeted delivery composition of claim 1, wherein said nanocarrier comprises a stealth agent.

4. The targeted delivery composition of claim 3, wherein said stealth agent is poly(ethylene glycol).

5. The targeted delivery composition of claim 1, wherein said therapeutic or diagnostic agent is embedded in, encapsulated in, or tethered to said nanocarrier.

6. The targeted delivery composition of claim 5, wherein said nanocarrier is a liposome.

7. The targeted delivery composition of claim 1, wherein said nanocarrier is a liposome selected from the group consisting of SUVs, LUVs and MLVs.

8. The targeted delivery composition of claim 1, wherein said nanocarrier comprises a therapeutic agent selected from the group consisting of doxorubicin, cisplatin, oxaliplatin, carboplatin, 5-fluorouracil, gemcitibine and a taxane.

9. The targeted delivery composition of claim 1, wherein said diagnostic agent is a radioactive agent, a fluorescent agent, or a contrast agent.

10. The targeted delivery composition of claim 1, wherein said diagnostic agent is a radioactive agent selected from the group consisting of ¹¹¹In-DTPA, ^{99m}Tc(CO)₃-DTPA, and ^{99m}Tc(CO)₃-ENPy2.

11. The targeted delivery composition of claim 1, wherein said diagnostic agent is a fluorescent agent.

12. The targeted delivery composition of claim 1, wherein said diagnostic agent is a MR agent or a X-ray contrast agent.

13. The targeted delivery composition of claim 1, wherein said attachment component comprises a functional group for covalent attachment to said nanocarrier.

14. The targeted delivery composition of claim 1, wherein said attachment component is a lipid.

15. The targeted delivery composition of claim 14, wherein said lipid is a phospholipid, glycolipid, sphingolipid, or cholesterol.

16. The targeted delivery composition of claim 1, wherein the A portion of said conjugate is present in a lipid bilayer portion of said nanocarrier.

17. The targeted delivery composition of claim 16, wherein said nanocarrier is a liposome.

18. The targeted delivery composition of claim 1, wherein n is a number sufficient to allow said targeting agent to extend beyond the surface of said nanocarrier.

19. The targeted delivery composition of claim 1, wherein n is between 1 and 20.

20. The targeted delivery composition of claim 1, wherein n from 4 to 12.

21. The targeted delivery composition of claim 1, wherein n is 4, 5, 6, 7, 8, 9, 10, 11 or 12.

22. The targeted delivery composition of claim 1, wherein T is an aptamer.

23. The targeted delivery composition of claim 1, wherein T is an aptamer that targets a site present on a receptor selected from the group consisting of MUC-1, EGFR, FOL1R, Claudin 4, MUC-4, CXCR4, CCR7, somatostatin receptor 4, Erb-B2 (erythroblastic leukaemia oncogene homologue 2) receptor, CD44 receptor, VEGF receptor-2 kinase, and nucleolin.

24. A conjugate having the formula:
A-[(EG)(P)]ₙ-T;
wherein,
A is an attachment component;
[(EG)(P)]ₙ is a linking group, wherein the subscript n is an integer from 1 to about 40; and
each EG is independently selected from a group consisting of triethylene glycol, tetraethylene glycol, pentaethylene glycol, hexaethylene glycol, heptaethylene glycol, and octaethylene glycol;
P is independently selected from a group consisting of phosphate and thiophosphate; and,
T is a targeting agent.

25. The conjugate of claim 24, wherein said attachment component comprises a functional group for covalent attachment to a nanocarrier.

26. The conjugate of claim 24, wherein said attachment component is a lipid.

27. The conjugate of claim 26, wherein said lipid is selected from the group consisting of a phospholipid, glycolipid, sphingolipid, and cholesterol.

28. The conjugate of claim 24, wherein n is between 1 and 20.

29. The targeted delivery composition of claim 24, wherein n is from 4 to 12.

30. The targeted delivery composition of claim 24, wherein n is 4, 5, 6, 7, 8, 9, 10, 11, or 12.

31. The conjugate of claim 24, wherein n is 8.

32. The conjugate of claim 24, wherein T is an aptamer.

33. A conjugate having the formula:
(DT)-[(EG)(P)]ₘ-T;
wherein,
DT is a diagnostic agent, a therapeutic agent, or a combination thereof;
[(EG)(P)]ₘ is a linking group, wherein the subscript m is an integer from 1 to about 40; and
each EG is independently selected from a group consisting of triethylene glycol, tetraethylene glycol, pentaethylene glycol, hexaethylene glycol, heptaethylene glycol, and octaethylene glycol;
P is independently selected from a group consisting of phosphate and thiophosphate; and,
T is a targeting agent.

34. The conjugate of claim 33, wherein said diagnostic agent is a radioactive agent, a fluorescent agent, or a contrast agent.

35. The conjugate of claim 33, wherein said diagnostic agent is a radioactive agent is selected from the group consisting of ¹¹¹In-DTPA, ^{99m}Tc(CO)₃-DTPA, and ^{99m}Tc(CO)₃-ENPy2.

36. The conjugate of claim 34, wherein said diagnostic agent is a fluorescent agent.

37. The targeted delivery composition of claim 33, wherein said diagnostic agent is a MR agent or a X-ray contrast agent.

38. The conjugate of claim 33, wherein said therapeutic agent is an anticancer agent selected from the group consisting of doxorubicin, cisplatin, oxaliplatin, carboplatin, 5-fluorouracil, gemcitibine and a taxane.

39. The conjugate of claim 33, wherein m is between 1 and 20.

40. The conjugate of claim 33, wherein T is an aptamer.

41. The targeted delivery composition of claim 33, wherein T is an aptamer that targets a site present on a receptor selected from the group consisting of MUC-1, EGFR, FOL1R, Claudin 4, MUC-4, CXCR4, CCR7, somatostatin receptor 4, Erb-B2 (erythroblastic leukaemia oncogene homologue 2) receptor, CD44 receptor, VEGF receptor-2 kinase, and nucleolin.

42. A method of preparing a targeted delivery composition, comprising attaching a nanocarrier including a therapeutic or diagnostic agent to a conjugate having the formula:
A-[(EG)(P)]ₙ-T;
wherein,
A is an attachment component for attaching said conjugate to said nanocarrier;
[(EG)(P)]ₙ is a linking group, wherein the subscript n is an integer from 1 to about 40; and
each EG is independently selected from a group consisting of triethylene glycol, tetraethylene glycol, pentaethylene glycol, hexaethylene glycol, heptaethylene glycol, and octaethylene glycol;
P is independently selected from a group consisting of phosphate and thiophosphate; and,
T is a targeting agent.

43. The method of claim 42, wherein said attachment component is a lipid.

44. The method of claim 43, wherein said lipid is a phospholipid, glycolipid, sphingolipid, cholesterol, or a cholesterol derivative.

45. The method of claim 42, wherein the A portion of said conjugate is present in a lipid bilayer portion of said nanocarrier.

46. The method of claim 45, wherein said nanocarrier is a liposome.

47. The method of claim 42, wherein n is between 1 and 20.

48. The targeted delivery composition of claim 42, wherein n is from 4 to 12.

49. The targeted delivery composition of claim 42, wherein n is 4, 5, 6, 7, 8, 9, 10, 11 or 12.

50. The method of claim 42, wherein T is an aptamer.

51. A method for treating or diagnosing a cancerous condition in a subject, comprising administering to said subject a targeted delivery composition of claim 1, wherein said therapeutic or diagnostic agent is sufficient to treat or diagnose said condition.

52. The method of claim 51, wherein T is an aptamer that targets a site present on a receptor selected from the group consisting of MUC-1, EGFR, Claudin 4, MUC-4, CCR7, somatostatin receptor 4, Erb-B2 (erythroblastic leukaemia oncogene homologue 2) receptor, CD44 receptor, VEGF receptor-2 kinase, and nucleolin.

53. The method of claim 51, wherein said nanocarrier has embedded in, encapsulated in, or tethered to an anticancer agent selected from the group consisting of doxorubicin, cisplatin, oxaliplatin, carboplatin, 5-fluorouracil, gemcitibine and a taxane.

54. A method of determining the suitability of a subject for a targeted therapeutic treatment, comprising administering to said subject a targeted delivery composition of claim 1, wherein said nanocarrier comprises a diagnostic agent, and imaging said subject to detect said diagnostic agent.

55. A method for delivering a therapeutic agent to a subject, comprising administering to said subject a conjugate of claim 33, wherein DT is a therapeutic agent.

56. A method of determining the suitability of a subject for a targeted therapeutic treatment, comprising administering to said subject a conjugate of claim 33, wherein DT is a diagnostic agent, and imaging said subject to detect said diagnostic agent.
